(19) United States
(12) Patent Application Publication
(10) Pub. No.: US 2015/0224211 A1
(43) Pub. Date: Aug. 13, 2015

(54) LEUKOCYTES AS DELIVERY CELLS FOR IMAGING AND DISEASE THERAPY

(71) Applicants: Deryl L. Troyer, (US); Stefan H. Bossmann, (US)

(72) Inventors: Deryl L. Troyer, Manhattan, KS (US); Stefan H. Bossmann, Manhattan, KS (US); Hongwang Wang, Manhattan, KS (US); Matthew T. Basel, Manhattan, KS (US); Tej B. Shrestha, Manhattan, KS (US); Sebastian Wendel, Manhattan, KS (US)

(21) Appl. No.: 14/694,511

(22) Filed: Apr. 23, 2015

Related U.S. Application Data
(63) Continuation-in-part of application No. PCT/US2013/070515, filed on Nov. 18, 2013.
(60) Provisional application No. 61/728,083, filed on Nov. 19, 2012.

(51) Int. Cl. A61K 49/00; A61K 9/127; A61K 47/46; A61K 41/00; A61K 31/65

(52) U.S. Cl. CPC A61K 49/0013; A61K 41/0061; A61K 31/65; A61K 47/46; A61K 9/1271; A61K 49/0041; A61K 49/0084

(57) ABSTRACT

Theranostic methods are described herein, which are useful in diagnosing and/or treating infection, inflammation, and/or cancer. The methods utilize naturally-occurring leukocytes for in situ photodynamic therapy and imaging, and for the delivery of targeted therapies to the infection, inflammation, and/or cancer.

### CROSS-REFERENCE TO RELATED APPLICATIONS

The present application is a continuation-in-part of PCT/US2013/070515, filed Nov. 18, 2013, which claims the priority benefit of U.S. Provisional Patent Application Ser. No. 61/728,083, filed Nov. 19, 2012, entitled NEUTROPHILS AS DELIVERY CELLS FOR IMAGING AND DISEASE THERAPY, each of which are incorporated by reference in its entirety herein.

### FEDERALLY SPONSORED RESEARCH OR DEVELOPMENT

This invention was made with government support under #NSF-CBET 933701 and #NSF-DMR 1242765, awarded by the National Science Foundation. The United States government has certain rights in the invention.

### BACKGROUND OF THE INVENTION

1. Field of the Invention

The present invention relates to targeted therapeutic methods for diagnosing and treating inflammation, infection, and/or cancerous tissue using leukocytes to deliver active agents to these foci.

2. Description of Related Art

Targeted, yet highly effective therapies for various diseases are intensively sought. For example, in cancer treatment, even though primary tumors can often be reduced or eliminated, the metastatic disease that subsequently occurs usually results in fatality. However, many of the recently developed targeted therapies are complicated, time-consuming, and expensive. In addition, there remains a need for targeted and effective therapies for infectious diseases, such as antibiotic-resistant infections, as well as various inflammatory conditions.

### SUMMARY OF THE INVENTION

The present invention is broadly concerned with methods for the in situ treatment and/or diagnosis of infection, inflammation, and/or cancerous tissue in a subject having cancerous tissue or tissue infected or inflamed by a pathogen. The method uses naturally-occurring leukocytes of the subject (i.e., neutrophils, monocytes/macrophages, lymphocytes, and mixtures thereof) and comprises (consists essentially or even consists of) optionally administering a photosensitizing agent to the subject; administering a luminogenic substrate comprising luminol to the subject; and optionally detecting light generated by the luminogenic substrate to thereby image the infection, inflammation, and/or cancerous tissue. Advantageously, the naturally-occurring leukocytes accumulate in and near the infection, inflammation, and/or cancerous tissue and secrete oxidative species, these oxidative species react with the luminogenic substrate to generate light, which can not only be detected as noted above, but can also cause damage and destruction of the pathogen or cancerous tissue. In addition, the photosensitizing agent, when present, is activated by the light generated by the luminogenic substrate and the activated photosensitizing agent enhances the damage and destruction of the pathogen or cancerous tissue. Thus, the inventive methods involve in situ generation of light (energy), and preferably exclude photodynamic therapy utilizing external or externally-generated light sources (e.g., lasers, LEDs, etc. inserted into the subject).

Targeted methods of treating infection, inflammation, and/or cancerous tissue in a subject having cancerous tissue or tissue infected or inflamed by a pathogen are also described herein. The methods comprise (consist essentially or even consist of) providing naturally-occurring leukocytes of the subject (i.e., neutrophils, monocytes/macrophages, lymphocytes, and mixtures thereof); and loading the naturally-occurring leukocytes with an active agent. Advantageously, the loaded leukocytes accumulate in and near the infection, inflammation, and/or cancerous tissue and release the active agent to thereby treat the infection, inflammation, and/or cancerous tissue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Luminol imaging and detection of mouse mammary tumors 4 days before they are palpable (at day 6);

FIG. 2 is demonstrates that ALA photodynamic therapy generated by an internal light source from neutrophils and luminol can attenuate tumors in a mouse breast cancer model;

FIG. 3 demonstrates that GGP-liposomes loaded with rhodamine-dextran at 1:10 (3 nM) successfully loaded into the neutrophils (red);

FIG. 4 shows imaging from non-pathogenic (K-12 strain) killed E. Coli filled with rhodamine isothiocyanate and incubated with mouse blood for one hour, followed by lysis and smears to confirm flow cytometry findings showing that neutrophils were loaded with the preloaded bacteria;

FIG. 5 shows images from the raw 264.7 cell line (MOUSE MONOCYTES) loaded with E. coli after 24 hours of incubation;

FIG. 6 is a graph showing the results from testing neutrophils loaded with Lactobacillus filled with doxycycline for their ability to reduce numbers of a strain of E. coli resistant to nalidixic acid;

FIG. 7 illustrates the process of eradication of an infectious agent using neutrophils loaded with non-pathogenic bacteria carrying an antibiotic or antiseptic;

FIG. 8 is a graph of the antiseptic loading into the non-pathogenic bacteria from Example 3;

FIG. 9 is a graph of the additional analysis of antiseptic loading;

FIG. 10 shows the qualitative analysis of the antiseptic loading into the non-pathogenic bacteria;

FIG. 11 is a graph analyzing the retention of CHX in the bacteria over time;

FIG. 12 is a graph of the loading efficiency into the neutrophils;

FIG. 13 is a graph demonstrating that the M. luteus loaded with CHX and delivered using neutrophils could efficiently kill F. necrophorum;

FIG. 14 is a graph demonstrating the viability of F. necrophorum treated with the neutrophil-based chlorhexidine (CHX) drug delivery system in vitro. A. RPMI medium, B. Unmodified neutrophils, C. Neutrophils loaded with empty M. luteus microparticles, D. Neutropils loaded with CHX containing M. luteus microparticles; and

FIG. 15 is a graph demonstrating efficacy of the system in a mouse model of liver abcesses, based upon fusobacterial load in the liver of mice treated with neutrophils containing chlorhexidine (CHX) contained in microparticles (killed micrococcus luteus). A. PBS only, B. unmodified neutrophils, C. neutrophils loaded with empty microparticles, and D. Neutrophils loaded with microparticles containing CHX.

### DETAILED DESCRIPTION

The present invention is concerned with imaging lesions (e.g., tumors) for early detection and diagnosis of disease as well as treatment of infection, inflammation, lesions, and/or tumors by in situ photodynamic therapy. The present invention is also concerned with targeted methods of delivering active agents to sites of infection, inflammation, lesions and/or cancerous tissue. The methods of the invention are applicable in treating and/or diagnosing (theranostics) cancer, as well as infections, inflammation, and other types of lesions. The methods are suitable for diagnosing and/or treating subjects having cancerous tissue (e.g., tumors, metastases, and cancer cells) or tissue infected or inflamed by a pathogen (e.g., any infectious microorganism, such as a virus, bacterium, prion, or fungus). In the context of infection, inflammation, and disease, the invention is particularly advantageous for treating antibiotic-resistant pathogens.

In one or more embodiments, methods for the in situ treatment and/or imaging/detection of infection, inflammation, and/or cancerous tissue in a subject are described herein. Methods according to these embodiments take advantage of the naturally-occurring leukocytes of the subject. The term leukocytes, as used herein, encompasses neutrophils, monocytes/macrophages, lymphocytes, and mixtures thereof. Neutrophils are among the most numerous white blood cells in peripheral blood, and are often “first responders” to infection, inflammation, and other lesions. Monocytes in contrast to neutrophils are larger (hence could carry a larger therapeutic payload per cell). They are not as numerous as neutrophils but nevertheless can be isolated in large numbers from peripheral blood (or loaded after IV administration of drug-containing particles). Monocytes respond more slowly to infections than neutrophils, and differentiate into macrophages when they escape into tissues. Monocytes infiltrate tumors and chronic infectious foci such as granulomas, so they could be especially valuable as delivery vehicles for subjects suffering from such conditions. The term “naturally-occurring” as used herein denotes that the cells are in their natural form, and have not been: amplified, transformed, transfected, and/or otherwise modified such as through genetic or epigenetic modifications (e.g., have not been artificially altered to include a transgene and/or to change the expression, activity, or function of genes or gene products). In other words, the naturally-occurring cells are in their native, non-modified form, which may include naturally-occurring mutations, but does not encompass artificially-imposed genetic alterations. In one or more embodiments, the naturally-occurring cells have not been cultured (in vitro or ex vivo), where the term “cultured” refers to growing (expanding) cells, such as on a cell culture plate or in a culture tube. This is in contrast to “incubating” cells under conditions to maintain live cells without growing or expanding the cells. In one or more embodiments, such naturally-occurring cells may be autologous to the subject. In one or more embodiments, the naturally-occurring cells are “circulating” cells (in the bloodstream) of the subject, which means they have not been: injected into the subject, removed from the subject, cultured, and/or re-injected into the subject. In other words, methods according to the invention preferably exclude steps of administering non-naturally-occurring or otherwise exogenous leukocytes to the subject as a source of in situ light.

For imaging and in situ photodynamic therapy, the methods comprise administering a luminogenic substrate to the subject. The luminogenic substrate is a chemical that exhibits chemiluminescence, and more particularly is luminol (5-Amino-2,3-dihydro-1,4-phthalazinedione). Other luminogenic substrates are isoluminol (6-amino-2,3-dihydrophthalazine-1,4-dione), ABEI (6-((4-aminobutyl)(ethyl)amino)-2,3-dihydrophthalazine-1,4-dione) and L-0123 (8-amino-5-chloro-7-phenyl-2,3-dihydropyrido[3,4-d]pyridazine-1,4-dione), as well as acridinium derivatives, such as MMAC (9((4-methoxyphenoxy)carbonyl)-10-methylacridin-10-ium) or 2, -methoxy-10-methyl-9-(phenoxycarbonyl)acridin-10-ium (Yamaguchi et al, Analytica Chimica Acta 665 (2010) 74-78). The luminogenic substrate is administered locally or systemically via intravenous injection, intraperitoneal injection, intramuscular injection, intratumoral injection, intraarterial injection, or a combination thereof to deliver the luminogenic substrate to the subject's blood stream. The luminogenic substrate is typically administered dispersed in a pharmaceutically-acceptable carrier. The term “carrier” is used herein to refer to diluents, excipients, and the like, in which the luminogenic substrate may be dispersed for administration. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would naturally be selected to minimize any degradation of the compound or other agents and to minimize any adverse side effects in the subject, as would be well known to one of skill in the art. Pharmaceutically-acceptable ingredients include those acceptable for veterinary use as well as human pharmaceutical use, and will depend on the route of administration. For example, compositions suitable for administration via injection are typically solutions in sterile isotonic aqueous buffer. Exemplary carriers for use with the luminogenic substrate include complexes with beta-cyclodextrin in phosphate-buffered saline (PBS), or mixtures with polyethylene or oligoethylene in PBS, and the like.

Advantageously, the naturally-occurring leukocytes in the subject's bloodstream accumulate in and near any infection, inflammation, and/or cancerous tissue and secret oxidative species. Preferably, the naturally-occurring leukocytes accumulate in and near the infection, inflammation, and/or cancerous tissue within about 2 to about 5 days after administration of the luminogenic substrate. Due to this accumulation, these oxidative species are secreted in a targeted manner in and near the site of infection, inflammation, and/or cancerous tissue, where they react with the luminogenic substrate in the bloodstream to generate light that can have anti-cancer, anti-bacterial, anti-fungal, and/or anti-viral effects (i.e., causes damage and destruction of the pathogen or cancerous tissue). For example, myeloperoxidase and NADPH-oxidase generated by neutrophil secretion and/or apoptosis in and near the cancerous tissue results in the emission of light (bioluminescence). This is because luminol is oxidized by the neutrophil-generated oxidative species (superoxide, hydrogen peroxide, hypochlorite), creating an unstable species that breaks down emitting (blue) light. This blue light can be detected by appropriate imaging devices for early tumor detection and diagnosis. Thus, in one aspect, the method further comprises detecting the light generated by the luminogenic substrate to thereby image the site of infection, inflammation, and/or cancerous tissue. It can also be used to excite other fluorophores by either light absorption or fluorescence resonance energy transfer (FRET), resulting in a red-shift of emission. This in situ photodynamic therapy can be used for infectious disease or cancer treatment. Since human tissue is most transparent between 600 and 1000 nm, transferring the excited state energy to an appropriate fluorophore results in the capability of detecting the fluorescence occurring from deep-seated tumors.

For the photodynamic treatment of pathogens it is particularly preferred that a photosensitizing agent (aka “photosensitizer”) first be administered to the subject before administering the luminogenic substrate. Preferably, the photosensitizing agent is administered about 2 days before administering the luminogenic substrate. The photosensitizing agent can be administered locally or systemically via intravenous injection, intraperitoneal injection, intramuscular injection, intratumoral injection, intraarterial injection, or a combination thereof. Furthermore, the photosensitizing agent can be delivered as a prodrug by means of the same delivery modalities. The photosensitizing agent can be administered dispersed in a pharmaceutically-acceptable carrier as described herein, with preferred carriers for the photosensitizing agent being PBS, complexes of the photosensitizer or prodrug with alpha-, beta- or gamma-cyclodextrin, adsorbed onto albumin in PBS or dissolved PBS/polyethylene glycol mixtures. The photosensitizing agent is selected such that its absorption spectrum permits activation based upon the emission spectrum of the luminogenic substrate. In one or more embodiments, the luminogenic substrate generates light of a first wavelength, which activates the photosensitizing agent, when present. Accordingly, the photosensitizing agent generates or emits light of a second wavelength, which can be detected to determine the location of the infection, inflammation, and/or cancerous tissue in the subject. The activated photosensitizing agent can not only be used for imaging, but also enhances the damage and destruction of the pathogen or cancerous tissue. Exemplary photosensitizing agents include metallated (e.g. Mg(II), Zn(II), Pd(II), Pt(II)) and non-metallated porphyrins (e.g. protoporphyrin IX), dihydroporphyrins (chlorins), and tetrahydroporphyrins (bacteriochlorins), hypericin, and ruthenium-polypyridinium complexes, as well as photosensitizer-generating prodrugs (e.g., 5-aminolevulinic acid (“ALA”)), and mixtures or combinations thereof. Prodrugs that result in the formation of photosensitizing agents in vivo are particularly preferred. One example is ALA, a precursor for protoporphyrin IX. ALA is the substrate for the biosynthesis of protoporphyrin IX in the mitochondria of cancer cells and pathogens. Administration of ALA results in the formation of protoporphyrin IX in vivo, which acts as the photosensitizer in the photodynamic treatment process. Combinations of one or more photosensitizing agents can also be used.

Regardless of the embodiment, the damage and/or destruction to the cancerous tissue or pathogen from the photodynamic therapy (via the luminogenic substrate alone or in combination with the photosensitizing agent) preferably results in apoptosis of the cancerous tissue or pathogen, as opposed to necrosis.

In one or more embodiments, targeted methods of treating infection, inflammation, and/or cancerous tissue in a subject are also described herein. The methods use leukocytes to deliver an active agent to the site of inflammation, infection, and or cancerous tissue in the subject. Advantageously, the leukocytes are naturally-occurring and are selected from the group consisting of neutrophils, monocytes, lymphocytes, and mixtures thereof. In one or more embodiments, the leukocytes are autologous to the subject. The leukocytes are loaded with the active agent, and accumulate in and near the site of infection, inflammation, and/or cancerous tissue, where they naturally release the active agent (either by apoptosis or secretion) to thereby treat the infection, inflammation, and/or cancerous tissue in a targeted manner. The terms “load,” “loading,” or “loaded,” refer to the process or feature of the active agent being taken up by the leukocyte (such as through phagocytosis or other, endocytosis) and engulfed or internalized by the cell, such that it ends up inside the leukocyte (i.e., on the other side of the cell membrane).

In one or more embodiments, the naturally-occurring leukocytes are circulating leukocytes, which have not been removed from the subject, cultured, injected or re-injected into the subject. In other words, the active agent is loaded into the leukocytes in vivo (in the bloodstream) without removing the leukocytes from the subject. More particularly, loading in this aspect, comprises administering the active agent to the subject, where it is preferentially taken up by the circulating leukocytes in vivo. The active agent can be dispersed in a pharmaceutically-acceptable carrier as described herein, with preferred carriers for the active agent being a solution in PBS, a complex with alpha-, beta-, or gamma-cyclodextrin in PBS, or solution in PBS and polyethylene glycol. The active agent can be administered locally or systemically via intravenous injection, intraperitoneal injection, intramuscular injection, intratumoral injection, intraarterial injection, or a combination thereof. As the loaded leukocytes continue to circulate, they ultimately accumulate in and near the foci of inflammation, infection, and/or cancerous tissue to deliver their payload (i.e., the active agent).

In one or more embodiments, the leukocytes are loaded ex vivo. In other words, a blood sample is collected from the subject under ex vivo conditions, and the leukocytes are loaded outside of the subject's body (i.e., outside of the bloodstream), but without isolating them from the blood (for example, via an extracorporeal shunt). This removes the need to aseptically remove and culture cells without somehow changing them during culture. The fact that leukocytes in peripheral blood can be quickly loaded without isolating and culturing them is a significant advantage of this invention. Thus, the leukocytes are not isolated from the blood sample, and the blood sample containing the leukocytes is simply incubated with the active agent. In general, the active agent is incubated with the blood sample until the leukocytes in the blood sample are well loaded. It will be appreciated that specific incubation times will vary depending upon the target leukocytes to be loaded. That is, neutrophils take up the active agents more quickly than other leukocytes, such as monocytes or lymphocytes. Thus, shorter incubation times are required for neutrophils, as opposed to other leukocytes. In general, the active agent will be incubated with the blood sample for less than about 12 hours, preferably from about 30 min to about 6 hours, and more preferably from about 30 minutes to about 1 hour.

Regardless, the leukocytes in the blood sample take up the active agent, and then the blood sample containing the loaded leukocytes is administered to (i.e., injected into) the subject. In one or more embodiments, the blood sample containing the loaded leukocytes can be dispersed in a pharmaceutically-acceptable carrier for administration to the subject. Although the inventive method is intended primarily for autologous leukocyte injections, it is contemplated that a blood sample collected from a first subject could be loaded ex vivo and then injected for targeted therapy into a second subject (e.g., who is otherwise a compatible recipient of such a blood sample). Regardless, the loaded leukocytes ultimately accumulate in and near the foci of infection, inflammation, and/or cancerous tissue and release the active agent to thereby treat the infection, inflammation, and/or cancerous tissue in the recipient subject. Preferably, the blood sample containing the loaded leukocytes is administered to (i.e., injected into) the subject less than about 24 hours after the blood sample has been collected, and more preferably less than about 12 hours after collection.

Regardless of the embodiment, it will be appreciated that the treatment methods of the invention are fast, highly efficient and extremely targeted drug delivery methods for lesion-causing diseases, including infectious diseases, inflammation, and cancer. These drug-delivery methods utilized lesion-homing leukocytes, which are abundant in peripheral blood. Autologous cells can also be used to avoid immune rejection.

In one or more embodiments, the active agent is loaded into the leukocytes using a delivery vehicle in which the active agent is encapsulated. The delivery vehicle will typically comprise a targeting moiety on the surface thereof for preferential uptake by the naturally-occurring leukocytes. This is particularly advantageous for in vivo loading of the active agent. Exemplary targeting moieties includes peptides, antibody fragments, and negative surface charges mimicking bacteria, and the like that are specific to one or more of the neutrophils, monocytes, and/or lymphocytes, and can be attached to the surface of the delivery vehicle for preferential uptake. In one or more embodiments, the delivery vehicle is selected from the group consisting of liposomes, polymersomes, supramolecular structures, vesicles, exosomes, and combinations thereof. The active agent is incubated with the delivery vehicle until encapsulated in the delivery vehicle (and preferably for about 1 hour to about 12 hours, and more preferably for about 1 to about 6 hours). The preloaded delivery vehicle can then be used to load the active agent into the leukocytes in vivo or ex vivo.

In one or more embodiments, the delivery vehicle is a non-pathogenic, inactivated bacteria. That is, the leukocytes can be quickly loaded via incubation with non-pathogenic, chemical or heat-inactivated bacteria that have been preloaded with the active agent(s). For example, loading leukocytes with traditional delivery vehicles (e.g., liposomes) outside the bloodstream should be done rapidly, given that leukocytes can only survive up to a maximum of 24 hours ex vivo. However, incubation in whole blood with pre-loaded, killed bacteria can be accomplished much more quickly enhancing viability of the leukocytes for ex vivo loading. For example, neutrophils can be loaded with pre-loaded bacteria in about an hour. In the method, the bacteria are first loaded with the active agent by culturing the bacteria under appropriate conditions with the active agent, and then the bacteria are heat or chemically inactivated. Blood is drawn from the subject, and the preloaded, inactivated bacteria are incubated with the peripheral blood sample (containing the unisolated leukocytes) until the leukocytes are well-loaded. As noted above, specific incubation times will vary depending upon the target leukocytes to be loaded. In general, the inactivated bacteria are incubated with the peripheral blood sample is incubated less than about 12 hours (preferably about 30 minutes to about 2 hours, and more preferably from about 30 min to about 1 hour). For neutrophils, the incubation time is preferably about 1 hour. Any free (unloaded bacteria) is then separated from the loaded leukocytes. For example, the blood is centrifuged to remove free bacteria, the pellet is re-suspended in phosphate buffered saline (PBS) or other media, then administered to (i.e., injected into) the subject. Monocytes can be loaded in similar fashion as described for neutrophils, except that incubation times would be longer since monocytes take up bacteria more slowly. In other words, monocytes still would not have to be separated or isolated from blood, but a centrifugation or magnetic separation step would be used to separate unloaded bacteria from the cells prior to re-infusion of the loaded cells into the subject.

Non-pathogenic, inactivated bacteria can also be used to load the leukocytes in vivo. In one or more embodiments, the bacteria can be opsonized before administering the loaded bacteria (via a pharmaceutically-acceptable carrier) to the subject for preferential uptake by the naturally-occurring leukocytes in vivo. In one or more embodiments, the preloaded bacteria is mixed with bacteria-specific antibodies, and this mixture is administered to the subject for preferential uptake by the naturally-occurring circulating leukocytes in vivo.

The approach of using bacteria as the delivery vehicle for loading the active agent into the leukocytes is suitable for use with most non-pathogenic bacteria strains. Non-limiting examples of suitable bacteria include those selected from the group consisting of Magnetospirillum, Lactobacillus, Micrococcus luteus, Escherichia coli (e.g., laboratory strains used for plasmid isolation), and the like.

A wide variety of active agents can be used in the inventive methods. Thus, the term “active agent” as used herein refers to any therapeutic or diagnostic agent capable of being loaded into a leukocyte. Non-limiting examples of various types of agents that one might desire to deliver to a site of inflammation, infection, and/or cancerous tissue, include anticancer or chemotherapeutic drugs, antimicrobial drugs, anti-fungal drugs, anti-viral drugs, and/or anti-inflammatory drugs. Thus, suitable active agents include small molecule drugs, chemotherapeutic drugs (e.g., doxorubicin, salinomycin, SN-38, cuculinin), fluorophores, photosensitizers (e.g., protoporphyrin IX, hypericin), antimicrobial agents (e.g., antibiotics, antibacterial agents, anti-viral agents, or anti-fungal agents, such as chlorhexidine, betadyne, gentamycin, tetracycline), anti-inflammatory agents (e.g., non-steroidal anti-inflammatory drugs, corticosteroids, curcumin, sulforaphane, and mixtures thereof), matrix metalloproteinase (MMP) inhibitors, MDR blockers (e.g., verapamil, resveratrol), biologics, and/or magnetic nanoparticles. In one or more embodiments, for even more targeted delivery, the active agent can also be tethered to nanomaterials such as dextran or dendrimers or magnetic nanoparticles by a caspase-cleavage sequence for targeted release only in the vicinity (proximate region) of the target tissue upon apoptosis of the leukocyte. These tethered active agents delivered via the leukocytes can serve as powerful hidden prodrugs to be released when the leukocytes undergo apoptosis. That is, the natural fate of leukocytes is to undergo apoptosis shortly after they have reached the foci of inflammation, infection, and/or cancer. Likewise, caspases are activated only by the process of apoptosis. Thus, the tethered drug will be released in active form only when the leukocytes undergo apoptosis and the tether is cleaved by the activated caspases. Hence, this is a highly specific way to deliver a powerful drug as an inactive prodrug specifically to a tumor or site of infection or inflammation using the lesion-homing leukocytes.

In one or more embodiments, the delivery vehicle itself can be chosen as being integral to the active agent. In particular, bacteria that make magnetic nanoparticles naturally (e.g., Magnetospirillum, ATCC 700264) can be used to load magnetic nanoparticles into the leukocytes, in order to generate cell-mediated magnetic hyperthermia in the tumors. These bacteria can be heat inactivated after making the magnetic nanoparticles and loaded into leukocytes for cell-mediated magnetic hyperthermia. Of course, cell-mediated magnetic hyperthermia can also be achieved by loading magnetic nanoparticles into the leukocytes via another suitable delivery vehicle (e.g., other bacteria or traditional delivery vehicle).

The targeted delivery methods can also be used to deliver a photosensitizer (e.g., protoporphyrin IX, hypericin, etc.), into a tumor for an alternative photodynamic therapy technique. Many photodynamic drugs can also red-shift the emitted light that would concurrently allow imaging of tumors deeper within the body, thus permitting theranostic applications. For example, the loaded leukocytes of the invention can also be used for in situ photodynamic therapy of cancer or microbial infections. In one embodiment of the invention, leukocytes are loaded with fluorophores that are released in active form when the leukocytes undergo apoptosis, shortly after reaching a lesion. Leukocyte-generated oxidative species cause bioluminescence of fluorophores, which can detected by appropriate imaging devices and can be utilized for early, deep-seated tumor detection.

It will be appreciated that the embodiments described herein utilize leukocytes, as natural homing devices for targeted therapy of cancer or infectious lesions. These abundant (preferably autologous) cells can be specifically loaded with an effective amount of therapeutic or diagnostic agents using delivery vehicles having targeting moieties attached thereto that are specific for the leukocytes, to create a stealth system for delivering therapeutic drugs such as for anti-cancer, anti-microbial and anti-inflammatory applications, hidden prodrugs for release at targeted sites, and cancer therapy for deep and disseminated tumors and cell-mediated magnetic hyperthermia. It will be appreciated that the “effective amount” of the agents will depend upon the particular agent used and the subject. In general an “effective amount” refers to the amount that will elicit the biological or medical response of a tissue, system, or subject that is being sought by a researcher or clinician, and in particular elicit some desired therapeutic effect as against the inflammation, infection, and/or cancerous tissue. Once the leukocytes are in the vicinity of the target tissue (tumor, lesion, etc.) the leukocytes undergo natural apoptosis, releasing their payload (i.e., the active agent). In the case of cancer, it will be appreciated that clinically relevant agents (e.g., DMXAA (5,6-Dimethylxanthenone-4-acetic Acid, ASA404, Vadimezan), cytokines, G-CSF, etc.) can be co-administered along with or prior to the loaded leukocytes to increase migration into tumors. Alternatively, a therapy causing necrosis in the tumor will increase infiltration of leukocytes.

The inventive methods offer minimal risk of immune rejection during cancer therapy, as autologous neutrophils are the first responders to tumors and other lesions, and the patient's own leukocytes can be used for the theranostic procedure.

It will be appreciated that therapeutic methods described herein are applicable to human subjects as well as any suitable non-human subject, including, without limitation, dogs, cats, and other pets, as well as, rodents, primates, horses, cattle, pigs, etc. The terms “therapeutic” or “treat,” and the like, as used herein, refer to processes that are intended to produce a beneficial change in an existing condition (e.g., infection, disease, condition) of a subject, such as by reducing the severity of the clinical symptoms and/or effects of the inflammation, infection, and/or cancer, and/or reducing the duration of the infection/symptoms/effects in the affected subject. The methods can be also applied for clinical research and/or study. Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Imaging and Photodynamic Therapy (PDT) of Tumors Based on Neutrophil Invasion

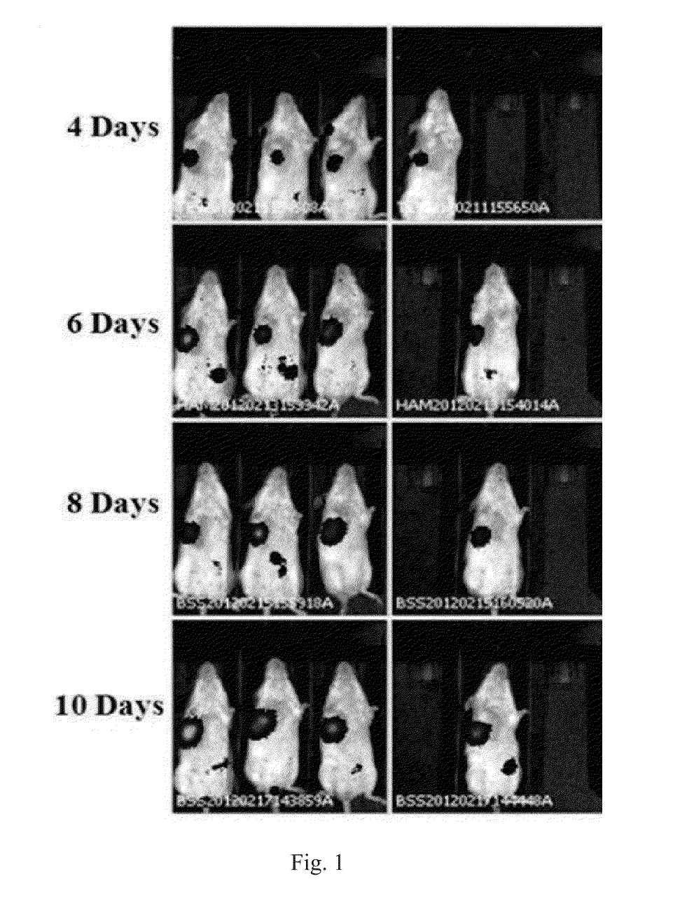

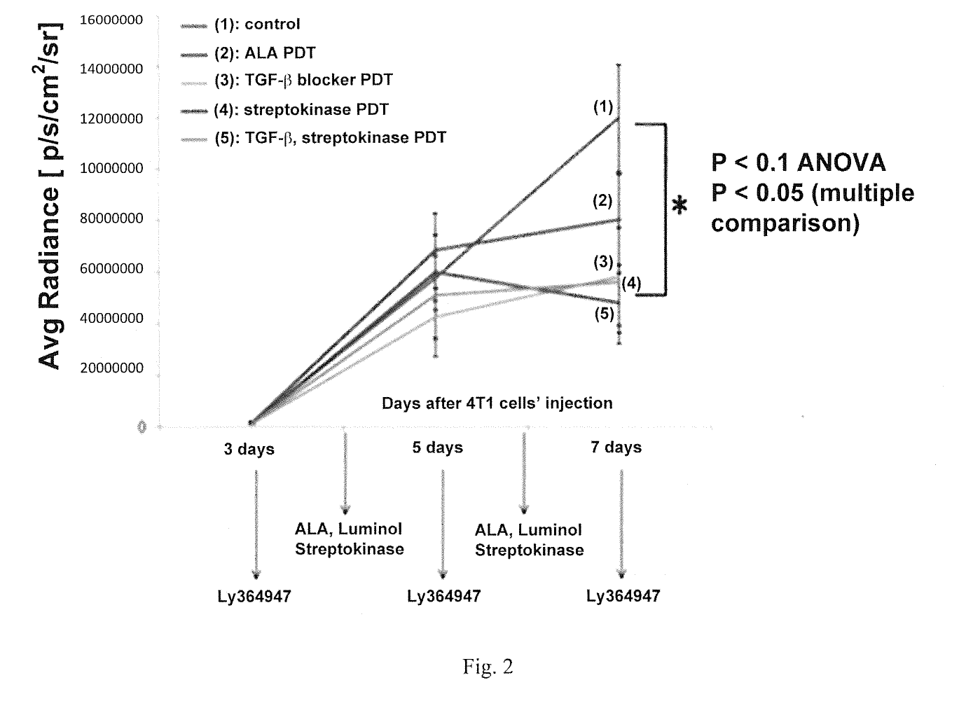

We tested the hypothesis that neutrophils secreting myeloperoxidase into tumors could be exploited to generate a light source that could be used for PDT. 1×10⁵ 4T1 mouse mammary carcinoma cells (ATCC) were transplanted into a mammary fat pad of a mouse. Four, six, eight and ten days after transplant, luminol (Sigma-Aldrich) was administered intrapertioneally (IP)(250 mg/kg) and the mouse was imaged on an IVIS Lumina imaging apparatus. The results are shown in FIG. 1. For in situ photodynamic therapy, the sensitizer alpha aminolevulinic acid (ALA), which is a precursor to protoporphyrin IX (a clinically used photosensitizer), was administered IP (100 mg/kg,) prior to luminol administration. The treatment was repeated, and four days after this internal, targeted, cell based PDT, there was a dramatic regression of the tumors as determined by imaging with luciferin (the tumors are engineered to express firefly luciferase). FIG. 2 shows this effect. Adjunct treatments designed to enhance the PDT effect included the administration of the TGF-β inhibitor LY34947 (1 mg/kg, IP), streptokinase (800 units/mouse, IV), or a combination of LY34947 and streptokinase.

### Example 2

### Loading of Peptide-Tagged Fluorescent Liposomes into Neutrophils

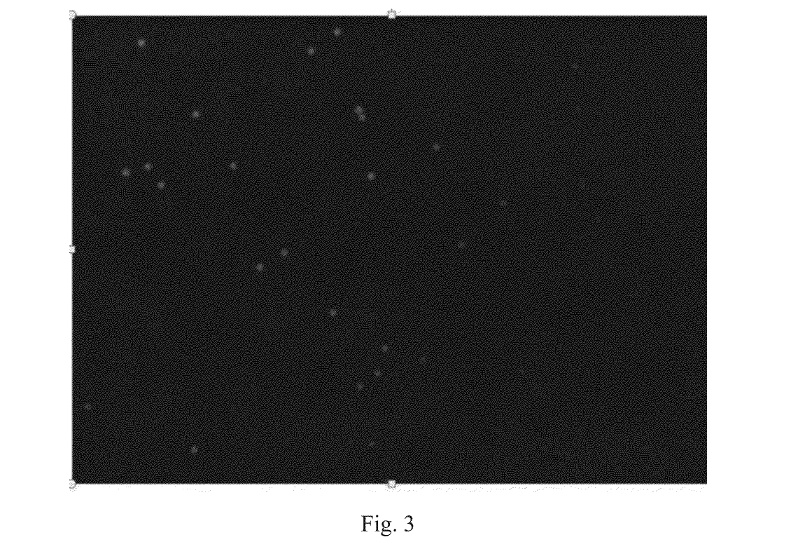

We have tested whether tumor-tropic neutrophils can be efficiently loaded without separation from the blood using liposomes with targeting peptides on their surface. The protocol is outlined below.
1—Liposomes tagged with a neutrophil-binding GGP peptide were constructed.
2—Liposomes were filled with rhodamine dextran.
3—Blood was collected.
4—The neutrophils can be isolated or the sample could be loaded into whole blood.
5—The blood was divided into three samples into 15 ml tubes: Control blood, Control GGP liposomes, and GGP liposomes.
6—Different dilutions of liposomes into neutrophils were tested:
   For example; 1:50, 1:30, 1:10 dilution (liposome volume:blood volume).
7—The liposomes were incubated with neutrophils for 2-3 hrs into the incubator at 37° C.
8—Lysis of red blood cells was started.
9—2-4 ml of lysis buffer was added to the samples.
10—The samples were spun at 350 ×g for 10 minutes.
11—Steps 8, 9, 10 were repeated three times.
12—The supernatant was discarded and the pellet was resuspended in 500 μl of 1×PBS.
13—20 μl of the cells were removed and mixed with trypan blue.
14—The white blood cells were counted.
15—After counting, 300 μl was taken for flow cytometry and diluted with 10 ml of 1×PBS.
16—200 μl of sample was taken for cytospin.
17—The cytospin slides were prepared and then:
   Fixed with 4% BNF, stained with Dapi, and mounted
   The slides were checked under the microscope.
      It will be appreciated that steps 8 et seq., above, would typically not be part of the clinical procedure but are included here to verify loading of the neutrophils. In the clinical setting, the blood containing the loaded neutrophils from step 7 would then be administered to (i.e., re-infused into) the patient. In some aspects, steps 8 et seq. could be carried out on small aliquots in the clinical setting to verify loading of the cells administered to the patient. FIG. 3 shows neutrophils loaded with rhodamine-dextran (a red-fluorescing dye) after incubation of mouse or bovine blood with liposomes targeted to them by the GGP peptide on the surface.

### Example 3

### Use of Nonpathogenic Bacteria as ‘Microparticles’ for Loading Defensive Cells

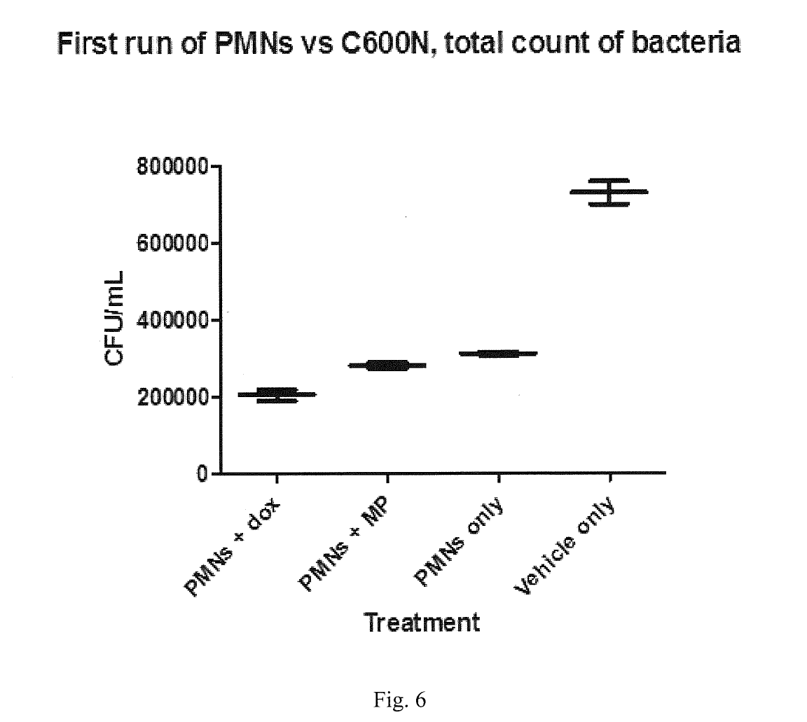

We have tested whether neutrophils loaded with non-pathogenic bacteria carrying doxycycline (an antibiotic) can be more effective than neutrophils alone in eliminating bacteria, and have found that this is indeed the case (FIG. 6). Some bacteria (K-12 strain E. coli) were purchased already filled with the fluorescent dye rhodamine isothyocyanate. Other nonpathogenic bacteria such as Lactobacillus acidophilus or Mycobacterium smegmatis have also been tested successfully for preloading with various therapeutic agents (anti-cancer agents or antimicrobial agents), then allowing defensive cells (neutrophils or monocytes) to phagocytose them. Below is a protocol for loading Lactobacillus with a tetracycline (doxycycline), and allowing uptake by neutrophils.
1—About 1×10⁸ cells/ml of bacteria was pipetting into a 15 ml tube.
2—The cells were spun at 3700×g for 15 minutes at 4° C.
3—The cells were washed with 1×PBS and spun again.
4—Step 3 was repeated three times.
5—The bacteria were loaded with Doxycycline (‘Dox’). Concentration of Dox: 200 ug/ml of Dox added to the resuspended bacteria from step 3.
6—The samples were mixed very well.
7—The tubes were then covered with foil.
8—The bacteria were incubated with Dox overnight at 4° C.
9—After incubation, the cells were spun at 3700×g for 15 minutes at 4° C.
10—The cells were washed with 1×PBS and spun again.
11—Step 10 was repeated three times.
12—2 ml of 1×PBS was added to the final pellet and mixed well.
13—Blood cells were collected for loading with bacteria.
14—20×10⁶ of bacteria cells were each added to 100 μl collected blood samples.
15—The samples were incubated for 1-2 hrs, spun down, and then resuspend in saline for administration to the patient.
   The following steps can be carried out for verification of loading (only an aliquot would be used in the clinical setting):
16—Lysis of the red blood cells was started, and 2-4 ml of lysis buffer was added to the samples
17—The samples were spun at 350×g for 10 minutes.
18—Steps 16 and 17 were repeated three times.
19—1 ml of 1×PBS was added to the final pellet and mixed gently.
20—20 μl of the cells were removed and mixed with trypan blue.
21—The white blood cells were counted.
22—Flow cytometry was run.

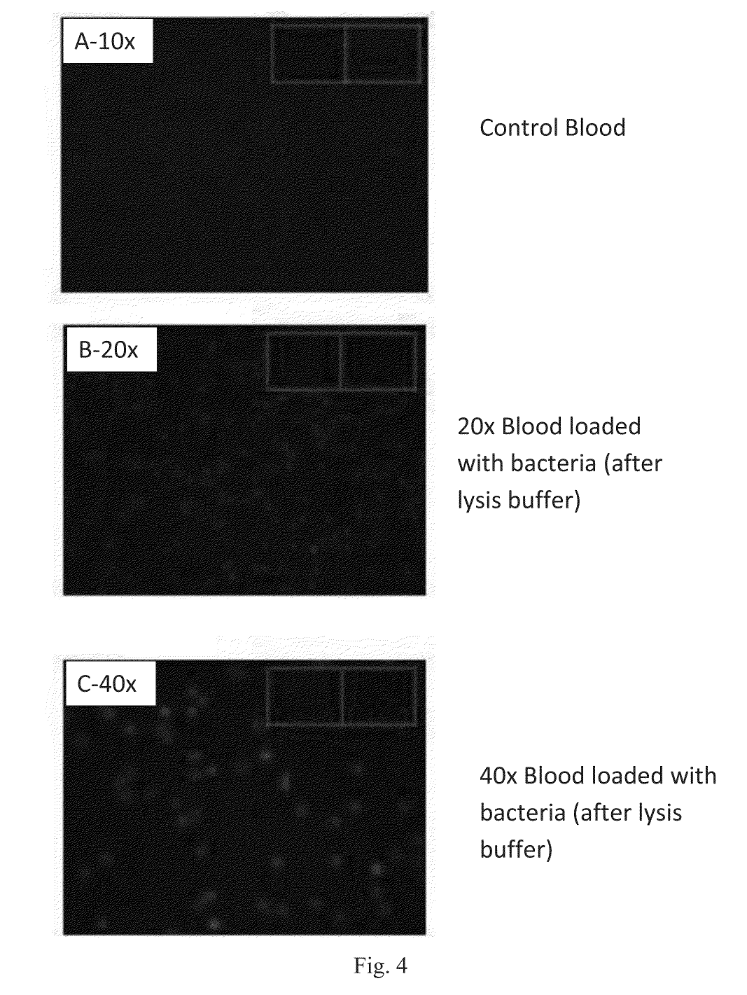

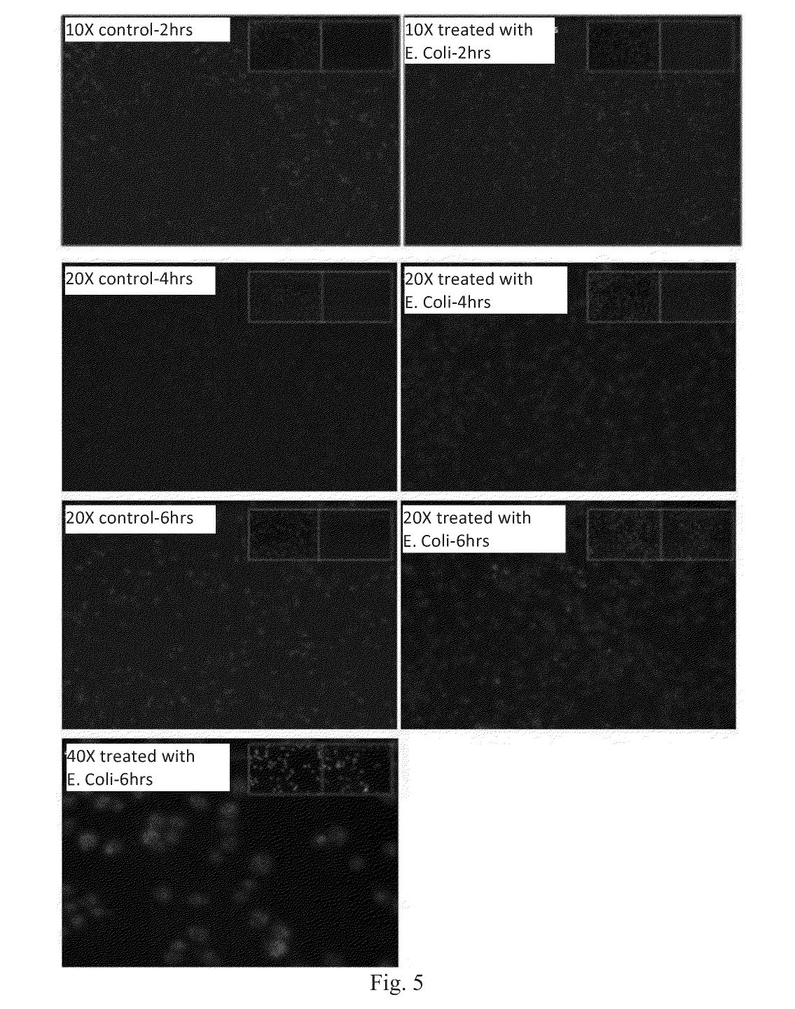

FIG. 4 shows mouse neutrophils loaded successfully with fluorescent dead non-pathogenic bacteria (K-12 strain E. coli) after 1 hr incubation in peripheral blood. FIG. 5 shows mouse monocytes in culture loaded with fluorescent bacteria.

In some embodiments, an in vitro test can be carried out to show that neutrophils loaded with antibiotic-filled lacobacilli can reduce numbers of bacterial colonies, as shown in FIG. 6. In this experiment, neutrophils loaded with Lactobacillus filled with doxycycline, neutrophils loaded with empty Lactobacillus, neutrophils not loaded with anything (“empty”), and vehicle controls were compared for their ability to reduce numbers of a strain of E. coli resistant to nalidixic acid. The PMNs filled with lactobacillus loaded with doxycycline significantly reduced numbers compared to PMNs alone (p<0.005) and PMNs loaded with empty Lactobacillus (p<0.05).

The protocol for bacterial enumeration following treatment with PMNs is outlined below.

### One to Two Days Before Experiment

1—5 ml of desired bacterial culture was started in the appropriate antibiotic concentration (e.g., one colony of bacteria in 5 ml of LB broth with appropriate antibiotic).
2—0.1 ml of overnight culture was spiked into 5 ml of fresh LB broth culture with appropriate antibiotic.
3—The cultures were grown until OD of approximately 0.3.
4—The bacteria was plated in 10⁻⁵, 10⁻⁶, and 10⁻⁷ dilutions to determine the number of bacteria per ml (CFU/mL). This was done to help in the later stages for determining the multiplicity of infection (MOI) when treating with PMNs. Everything was plated in duplicate at least, if not triplicate.

### The Day Before Experiment

1—Mouse plasma samples were pooled and readied.
2—The pooled plasma/serum samples were passed through a 0.22 micron filter.
3—The filtered plasma/serum samples were heated at 56° C. for 30 minutes to inactivate the complement pathway.
4—The samples were stored at 4° C. overnight.
5—5 ml of desired bacterial culture was started in appropriate antibiotic concentration (one colony of bacteria in 5 ml of LB broth with appropriate antibiotic)

### Day of the Experiment

1—0.1 ml of overnight culture was spiked into 5 ml of fresh LB broth culture with appropriate antibiotic.
2—The culture was grown until OD of approximately 0.3.
3—The bacteria were diluted to achieve 1×10⁻⁷ CFU/ml.
4—The bacteria were plated at 10⁻⁵ and 10⁻⁶ dilutions to approximate the correct number of bacteria (duplicate/triplicate plating).
5—1×10⁻⁷ bacteria were plated with 10% serum (final volume) for 20 minutes at 37° C. to opsonize the bacteria. (e.g., if starting with 1 ml of bacteria, use 110 μl of serum to achieve a final volume of 10% serum.) The opsonized bacteria were placed on ice.
6—10 μl of PMNs and all other samples obtained from a different lab were plated on plain LB agar to ensure that there was no contamination.
7—Add equal amounts of bacteria was added to the PMNs (1:1 MOI and 1:1 volume of each)
8—The samples were incubated at 37° C. for 90 minutes.
9—At the end of 90 minutes, the tubes were spun at 5,000 RPM for 5 minutes.
10—The supernatant was discarded and the pellet was resuspended in 100 μl of 1×PBS.
11—100 μl of 1% Triton X-100 (1% in 1×PBS) was added to achieve a final concentration of 0.5% Triton X-100 in the resuspended pellet. The sample was passed gently through a pipette 3-4 times ensuring that no bubbles were formed.
12—The prepared sample was allowed to sit at room temperature for 5 minutes for the Triton to lyse eukaryotic cells.
13—800 μl of LB broth was then added to bring up the final volume to 1 ml.
14—The samples were serially diluted and plated on LB agar plates with appropriate antibiotics. Everything was plated in duplicate at least.
15—The colonies were counted the next day for bacterial enumeration.

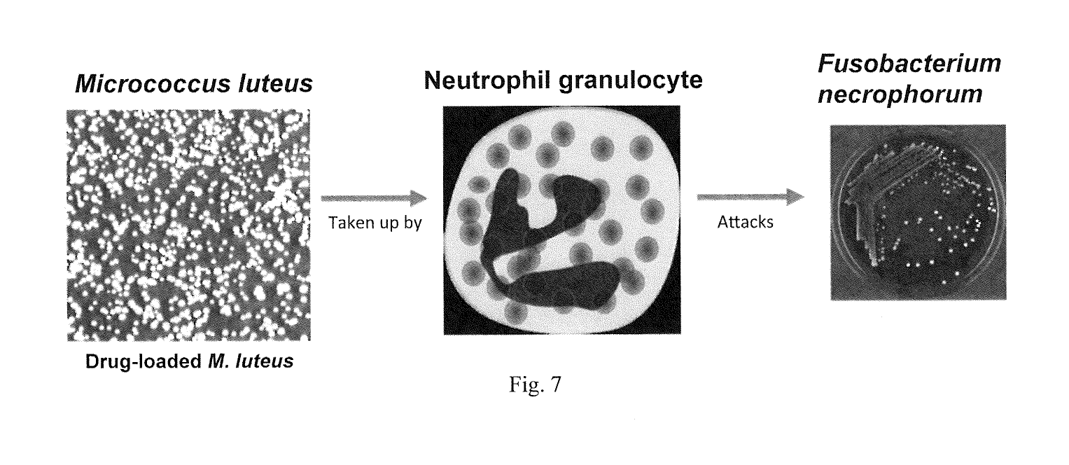

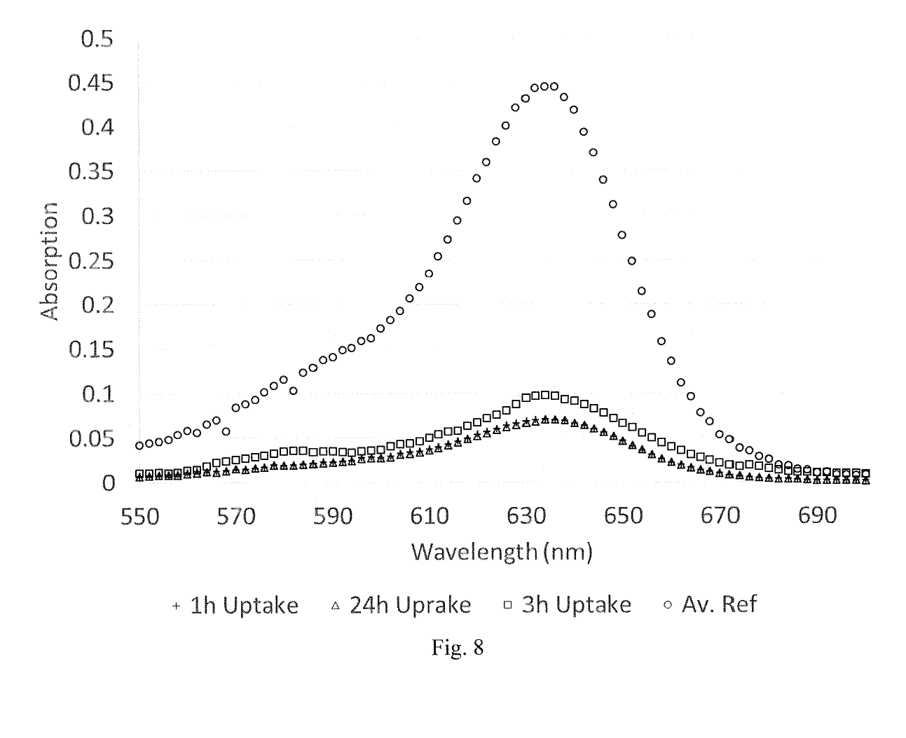

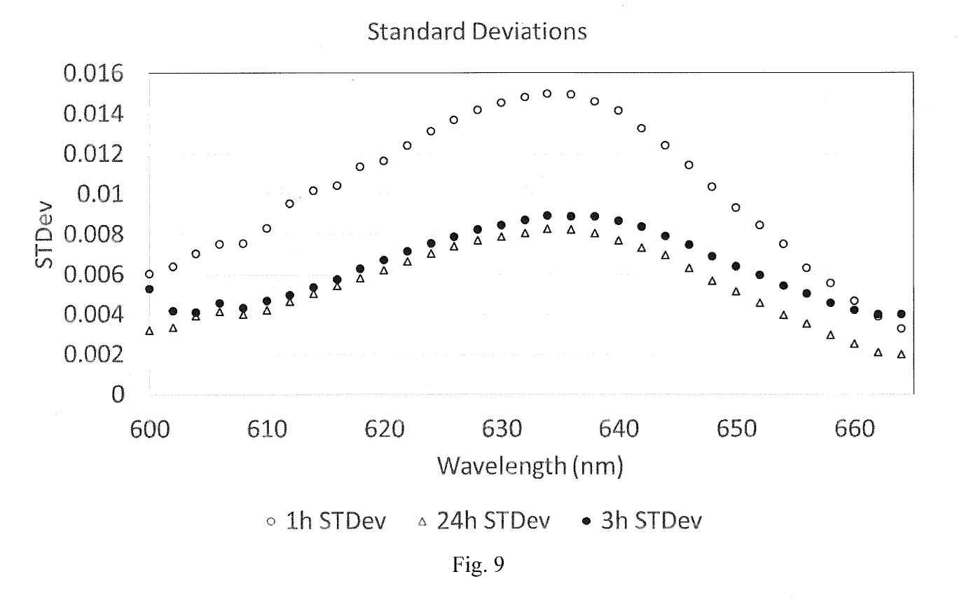

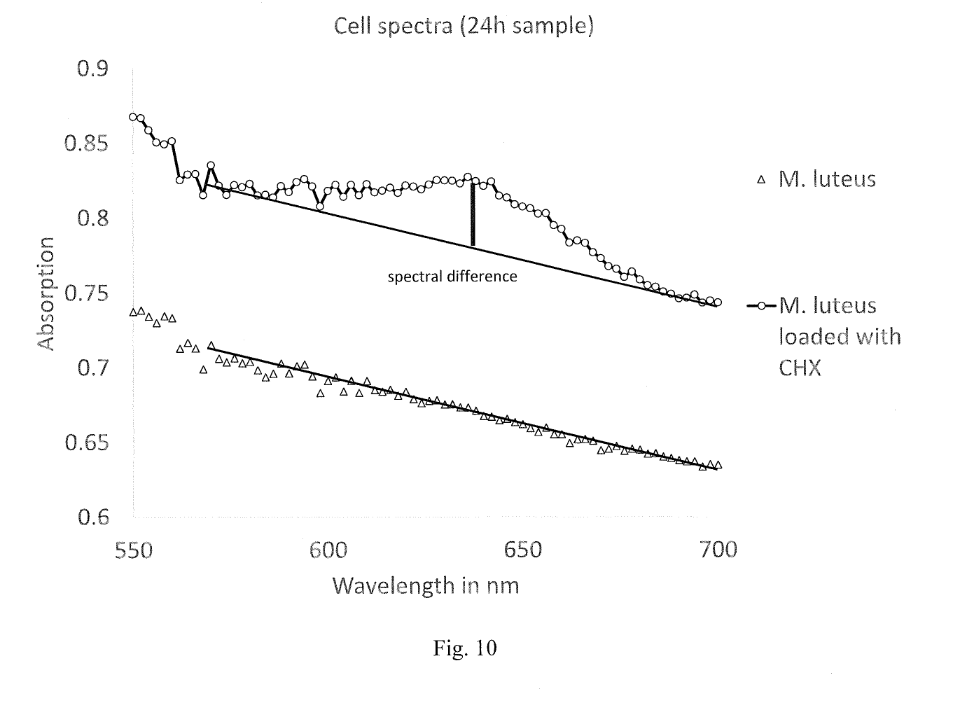

This protocol has been repeated using chlorhexidine (an example of a broad spectrum antiseptic) and Micrococcus luteus, which is a gram positive, spherical bacteria that inhabits soil, dust, water and is part of normal human skin flora. This process is illustrated in FIG. 7. Each experiment was repeated ten times and the results are shown in FIG. 8. The curves displayed are the average spectra of the respective experiment group. The results indicate that 16%-18% of the CHX provided were loaded into cells, where the 1 h-3 h comparison was not significant (p>0.3), but the 24 h-3 h comparison was significant (p<0.05). Further analysis is depicted in FIG. 9. FIG. 10 shows the direct measurement of CHX only for qualitative purposes because the background signal from cells make quantification of CHX via direct UV Vis measurement inaccurate. The spectral difference equals approximately 0.05, with an indirectly measured value of ˜0.07.

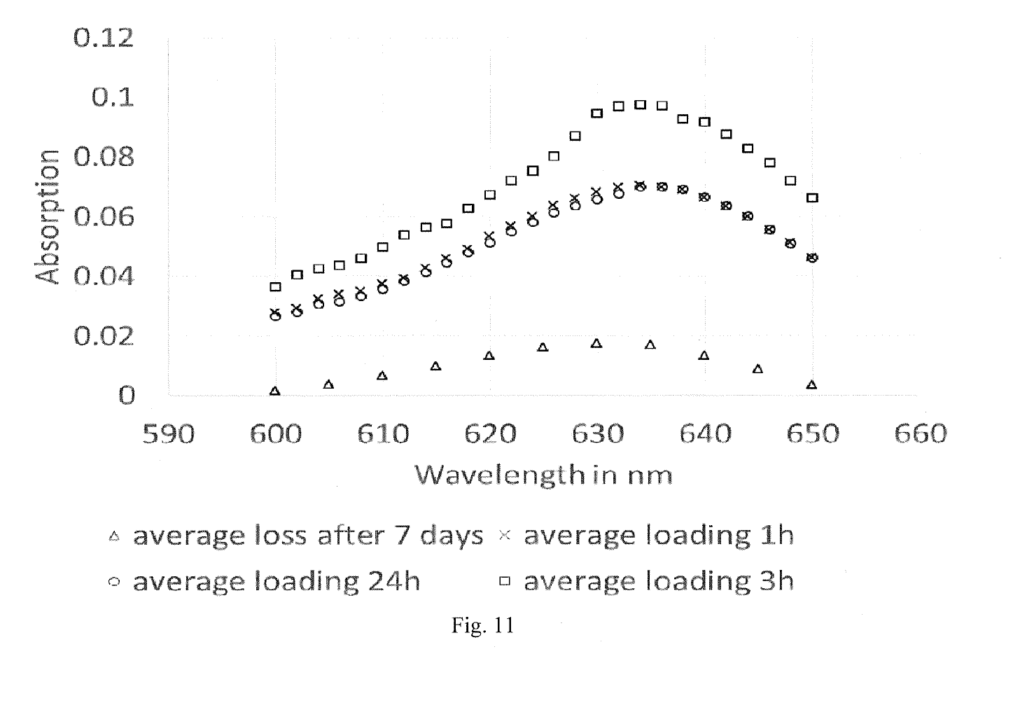

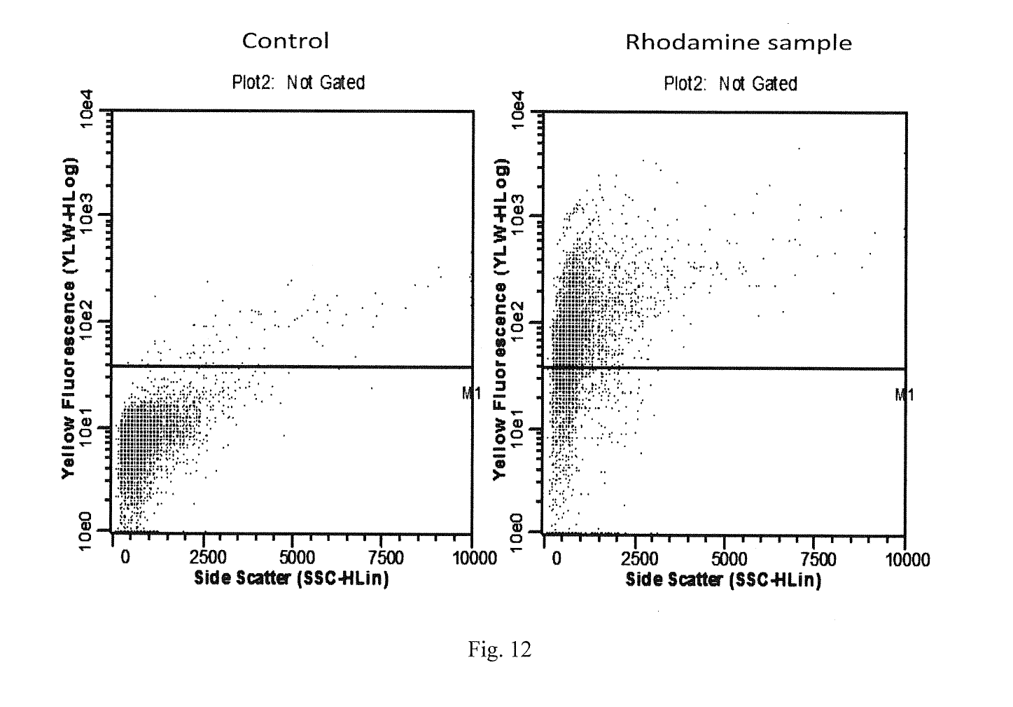

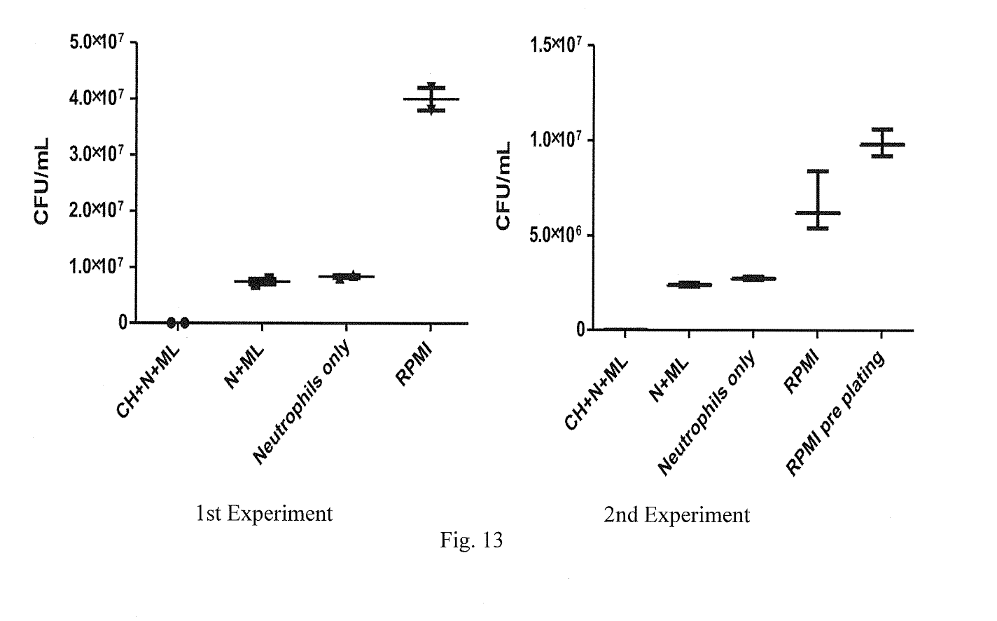

To determine the retention of CHX in the bacteria, samples were placed on a shaker for 7 days and the supernatant was measured to determine the amount of CHX that has left the cells. The results are shown in FIG. 11 and indicate that M. luteus can be loaded with CHX reliably and retains it to 80% over the course of 7 days. Next, the loading efficiency into the neutrophils was tested. Rhodamine dye was used as a marker to determine how many of the neutrophils provided were loaded. The results are shown in FIG. 12, which demonstrates that 70% of the neutrophils provided were loaded. In vitro testing was then carried out, as described above, using F. necrophorum to determine whether the M. luteus loaded with CHX and delivered using neutrophils could efficiently kill F. necrophorum. The delivery system utilizing CHX-modified M. luteus and neutrophil granulocytes eradicates F. necrophorum in vitro to 100%. The results are shown in FIG. 13.

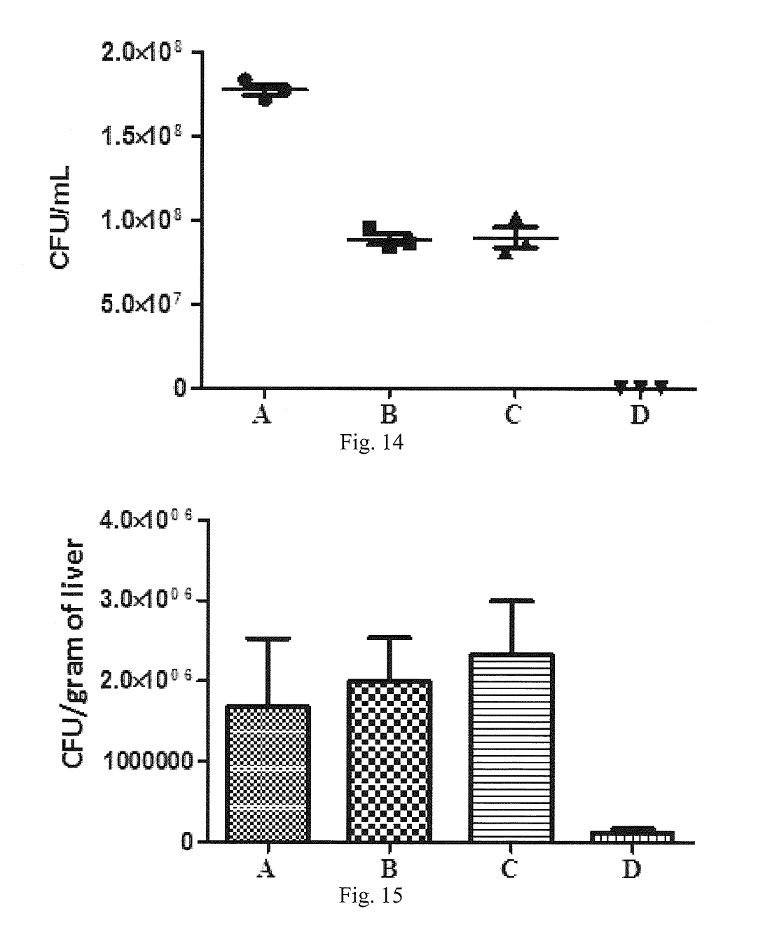

The experiment was repeated with results being shown in FIG. 14. These results reinforce the effectiveness of the system, where the M. luteus microparticles loaded with CHX and delivered using neutrophils completely eradicated the bacteria.

### Example 4

### In Vivo Mouse Studies

In a preliminary safety study, BALB/c mice (10-wk old; total 6) were injected intravenously (tail vein) with one million syngeneic neutrophils loaded with an average of 20 cells of M. luteus modified with either 1% or 2% chlorhexidine gluconate (CHX) (CHX microparticles). The animals were observed for 5 days post challenge to determine whether they demonstrated any clinical signs of toxicity. At the end of 5 days, the mice were euthanized and their kidneys, liver, brain, lung, spleen and heart were fixed in 10% formalin for histopathological evaluation. After clinical observation for one week, mice were euthanized. No gross or microscopic lesions were present in the liver, spleen, kidneys, heart, skeletal muscles, pancreas, brain or the site of intravenous administration. Spectroscopic analysis of the mouse liver revealed no residual CHX.

In an efficacy study that followed, Fusobacterium necrophorum subsp. necrophorum strain 8L1 was grown overnight from a single colony in PRAS-BHI. 0.3 ml of the starter culture was added to 10 ml of fresh PRAS-BHI broth and grown to an O.D.₆₀₀ of 0.7. 1 ml of this culture was diluted 1:40 to achieve a final concentration of approximately 4·10⁶ CFU/ml. 400 μl of the diluted bacteria was injected intraperitonially into 10 week old BALB/c mice. The mice were observed for two days before they were treated with neutrophils carrying antimicrobial cargo or controls.

Ten-week old BALB/c mice were randomly assigned into 6 groups with 9 mice per group. On day zero, all mice were infected intraperitonially with an infectious dose (approximately 1×10⁷ CFU) of F. necrophorum. On day 3, mice were injected (via tail vein) with 100 μl PBS (control), unmodified neutrophils, neutrophils carrying microparticles (an average of 20 cells of M. luteus loaded with chlorhexidine), or neutrophils containing unmodified, heat deactivated M. luteus. The animals were monitored for clinical signs for 5 days after treatment and were euthanized if any clinical signs developed. The mice were euthanized 5 days post treatment and all mice were examined post mortem for abscesses in the livers. Livers of the mice were weighed and homogenized in a tissue homogenizer for 1 min in modified lactate (ML) broth.

While none of the mice treated with neutrophils carrying CHX developed liver abscesses, at least two mice in the other groups developed gross or microabscesses. The most severe was the PBS only group, where 5 out of 9 mice developed abscesses. F. necrophorum bacterial load in the liver homogenate was significantly lower in mice treated with neutrophils loaded with microparticles containing CHX (FIG. 15). Spectroscopic analysis revealed no residual CHX in the liver homogenate. In summary, a very small amount of CHX (60 μg in 1.3×10⁶ neutrophils) was effective against the hepatic disease caused by F. necrophorum.

We claim:

1. A method for the in situ treatment and/or diagnosis of infection, inflammation, and/or cancerous tissue in a subject using naturally-occurring leukocytes of said subject, wherein said subject has cancerous tissue or tissue infected or inflamed by a pathogen, said method comprising:
optionally administering a photosensitizing agent to said subject;
administering a luminogenic substrate to said subject, wherein said naturally-occurring leukocytes accumulate in and near said infection, inflammation, and/or cancerous tissue and secret oxidative species, said oxidative species reacting with said luminogenic substrate to generate light and cause damage and destruction of said pathogen or cancerous tissue; and
optionally detecting said light generated by said luminogenic substrate to thereby image said infection, inflammation, and/or cancerous tissue,
wherein said photosensitizing agent, when present, is activated by said light generated by said luminogenic substrate and said activated photosensitizing agent enhances the damage and destruction of said pathogen or cancerous tissue.

2. The method of claim 1, wherein said luminogenic substrate is selected from the group consisting of luminol, isoluminol, 6-((4-aminobutyl)(ethyl)amino)-2,3-dihydrophthalazine-1,4-dione, and 8-amino-5-chloro-7-phenyl-2,3-dihydropyrido[3,4-d]pyridazine-1,4-dione, and acridinium derivatives.

3. The method of claim 1, wherein said photosensitizing agent is aminolevulinic acid, wherein said luminogenic substrate is administered to said subject about 2 days after administering said photosensitizing agent.

4. The method of claim 1, wherein said naturally-occurring leukocytes accumulate in and near said infection, inflammation, and/or cancerous tissue within about 2 to about 5 days after administration of said luminogenic substrate.

5. The method of claim 1, wherein said luminogenic substrate is administered via intravenous injection, intraperitoneal injection, intramuscular injection, intratumoral injection, intraarterial injection, or a combination thereof.

6. The method of claim 1, wherein said luminogenic substrate generates light of a first wavelength, said light of a first wavelength activating the photosensitizing agent, when present, said photosensitizing agent emitting light of a second wavelength, said method further comprising:
detecting said light of a second wavelength emitted from said photosensitizing agent to determine the location of said infection, inflammation, and/or cancerous tissue in said subject.

7. The method of claim 1, wherein said naturally-occurring leukocytes are circulating leukocytes that have not been: injected into said subject; removed from said subject; cultured; or re-injected into said subject.

8. A targeted method of treating infection, inflammation, and/or cancerous tissue in a subject, wherein said subject has cancerous tissue or tissue infected or inflamed by a pathogen, said method comprising:
providing naturally-occurring leukocytes of said subject, wherein said leukocytes are selected from the group consisting of neutrophils, monocytes, lymphocytes, and mixtures thereof; and
loading said naturally-occurring leukocytes with an active agent;
wherein said loaded leukocytes accumulate in and near said infection, inflammation, and/or cancerous tissue and release said active agent to thereby treat said infection, inflammation, and/or cancerous tissue.

9. The method of claim 8, wherein said naturally-occurring leukocytes are circulating leukocytes that have not been: injected into said subject; removed from said subject; cultured; or re-injected into said subject.

10. The method of claim 9, wherein said loading comprises administering said active agent to said subject, wherein said active agent is encapsulated in a delivery vehicle for preferential uptake by said naturally-occurring leukocytes in vivo.

11. The method of claim 10, wherein said delivery vehicle comprises a targeting moiety on the surface thereof for preferential uptake by said naturally-occurring leukocytes.

12. The method of claim 10, wherein said delivery vehicle is selected from the group consisting of liposomes, polymersomes, supramolecular structures, vesicles, and exosomes.

13. The method of claim 10, wherein said delivery vehicle is a non-pathogenic, inactivated bacteria.

14. The method of claim 13, wherein said bacteria is selected from the group consisting of Magnetospirillum, Lactobacillus, Micrococcus, and E. coli.

15. The method of claim 13, wherein said active agent is incubated with said bacteria and encapsulated therein prior to inactivation of said bacteria.

16. The method of claim 13, wherein said bacteria is opsonized prior to administering said active agent encapsulated in said delivery vehicle to said subject for preferential uptake by said naturally-occurring leukocytes.

17. The method of claim 8, wherein:
said providing comprises collecting a blood sample from said subject under ex vivo conditions, said blood sample comprising said naturally-occurring leukocytes, wherein said naturally-occurring leukocytes are not isolated from said blood sample; and
said loading comprises incubating said blood sample with said active agent, wherein said active agent is encapsulated in a delivery vehicle for preferential uptake by said naturally-occurring leukocytes in said blood sample;
said method further comprising:
injecting said blood sample comprising loaded leukocytes back into said subject, wherein said loaded leukocytes accumulate in and near said infection, inflammation, and/or cancerous tissue and release said active agent to thereby treat said infection, inflammation, and/or cancerous tissue in said subject.

18. The method of claim 17, wherein said delivery vehicle is selected from the group consisting of liposomes, polymersomes, supramolecular structures, vesicles, and exosomes.

19. The method of claim 17, wherein said delivery vehicle is a non-pathogenic, inactivated bacteria.

20. The method of claim 19, wherein said bacteria is selected from the group consisting of Magnetospirillum, Lactobacillus, Micrococcus, and E. coli.

21. The method of claim 19, wherein said active agent is incubated with said bacteria and encapsulated therein prior to inactivation of said bacteria.

22. The method of claim 17, wherein said active agent is incubated with said blood sample for about 1 to about 12 hours.

23. The method of claim 17, wherein said blood comprising said loaded leukocytes is injected back into said subject less than about 12 hours after collecting said blood sample from said subject.

24. The method of claim 8, wherein said active agent is selected from the group consisting of small molecule drugs, chemotherapeutic drugs, fluorophores, photosensitizers, antimicrobial agents, anti-inflammatory agents, matrix metalloproteinase (MMP) inhibitors, MDR blockers, biologics, magnetic nanoparticles, and combinations thereof.
